(19) United States
(12) Patent Application Publication
(10) Pub. No.: US 2025/0245833 A1
(43) Pub. Date: Jul. 31, 2025

(54) SLEEP STATE MEASUREMENT SYSTEM, SLEEP STATE MEASUREMENT METHOD, AND SLEEP STATE MEASUREMENT PROGRAM

(71) Applicant: SHIMADZU CORPORATION, Kyoto-shi (JP)

(72) Inventors: Koki YOSHIDA, Kyoto-shi (JP); Masafumi FURUTA, Kyoto-shi (JP); Tomotaka NAGASHIMA, Kyoto-shi (JP); Akie SOTOGUCHI, Kyoto-shi (JP); Ayako AKAZAWA, Kyoto-shi (JP); Mitsuki SAKAMOTO, Kyoto-shi (JP); Shima OKADA, Takatsuki-shi (JP); Masamitsu KAMON, Kyoto-shi (JP)

(21) Appl. No.: 18/854,421

(22) PCT Filed: Apr. 4, 2023
(86) PCT No.: PCT/JP2023/013983

§ 371 Date: Oct. 4, 2024

(30) Foreign Application Priority Data
Apr. 6, 2022 (JP) 2022-063542

(51) Int. Cl. G06T 7/00; G06T 7/246; G06T 7/73

(52) U.S. Cl. CPC G06T 7/0016; G06T 7/248; G06T 7/74; G06T 2207/30104; G06T 2207/30201; G06T 2207/30204

(57) ABSTRACT

Provided is a non-contact sleep state measurement system 100 capable of accurately measuring a sleep state regardless of age, disease, or the like of a subject. The sleep state measurement system includes: a frame image acquisition unit that acquires frame images including a subject P during sleep in time series; a difference information calculation unit that calculates difference information that is information indicating a difference between two frame images at different times; a subject attribute information acquisition unit that acquires subject attribute information that is information indicating an attribute of the subject; and a sleep state related information calculation unit that calculates sleep state related information that is information related to a sleep state of the subject using the difference information or secondary information obtained therefrom as an explanatory variable according to the subject attribute information.

### TECHNICAL FIELD

The present invention relates to a sleep state measurement system that measures a sleep state of a subject in a non-contact manner.

### BACKGROUND ART

Sleep has a deep relationship with the maintenance and growth/development of brain and body functions, and in recent years, there is an increasing need to grasp the state of sleep on a daily basis. On the other hand, sleep disorder such as insufficient sleep and insomnia has been regarded as a problem. In particular, sleep in childhood is important because it is said to affect not only the growth of the subject but also the lifestyle after adulthood.

A general screening/diagnosis of a sleep disorder is based on measurement with a wearable device that puts a heavy burden on a patient and grasping of a sleep state through a medical interview in a sleep clinic.

The measurement by the wearable device has a high burden on the patient, and there is also a risk that sleep data similar to that in normal times cannot be obtained. In addition, in the interview, since the patient himself/herself cannot report his/her sleeping state, the information is from a caregiver, a spouse, or the like, but it is difficult to make an accurate determination with this.

Therefore, in recent years, there has been a demand for a non-contact sleep state measurement system that can be easily installed and measured at home or the like. In Patent Literature 1, a parameter related to body motion (body motion amount, body motion duration, stationary duration, and the like) is calculated from a difference between image frames of a moving image obtained by capturing an image of a subject who is sleeping with a camera, and a sleep depth is estimated by machine learning on the basis of these parameters.

### CITATION LIST

### Patent Literature

Patent Literature 1: JP 2018-164615A

### SUMMARY OF INVENTION

### Technical Problem

However, in the conventional sleep depth estimation model, the age and the like of the subject are not considered, and there is room for improvement in accuracy.

The present invention has been made by focusing on this point for the first time, and an object of the present invention is to provide a non-contact sleep state measurement system capable of accurately measuring the sleep state regardless of the age or the like of the subject.

### Solution to Problem

The sleep state measurement system includes a frame image acquisition unit that acquires frame images including a subject during sleep in time series, a difference information calculation unit that calculates difference information that is information indicating a difference between two frame images at different times, a subject attribute information acquisition unit that acquires subject attribute information that is information indicating an attribute of the subject, and a sleep state related information calculation unit that calculates sleep state related information that is information related to a sleep state of the subject using the difference information and the subject attribute information as an explanatory variable.

### Advantageous Effects of Invention

According to the above configuration, since the sleep state is measured using the subject attribute information such as age as an explanatory variable in addition to being able to perform measurement in a natural sleep state without a burden on the subject, the measurement accuracy can be improved as compared with the related art.

### BRIEF DESCRIPTION OF DRAWINGS

FIG. 1 is an overall schematic diagram of a sleep depth measurement system according to an embodiment of the present invention.

FIG. 2 is a functional block diagram of the information processing apparatus according to the embodiment.

FIG. 3 is an explanatory diagram for explaining correction processing of difference information according to the embodiment.

FIG. 4 is a flowchart for explaining an operation of the sleep depth measurement system of the embodiment.

FIG. 5 is an operation explanatory diagram of a sleep depth measurement system according to another embodiment of the present invention.

FIG. 6 is an operation explanatory diagram of the sleep depth measurement system of the embodiment.

### DESCRIPTION OF EMBODIMENTS

Hereinafter, an embodiment of the present invention will be described with reference to the drawings.

### First Overview

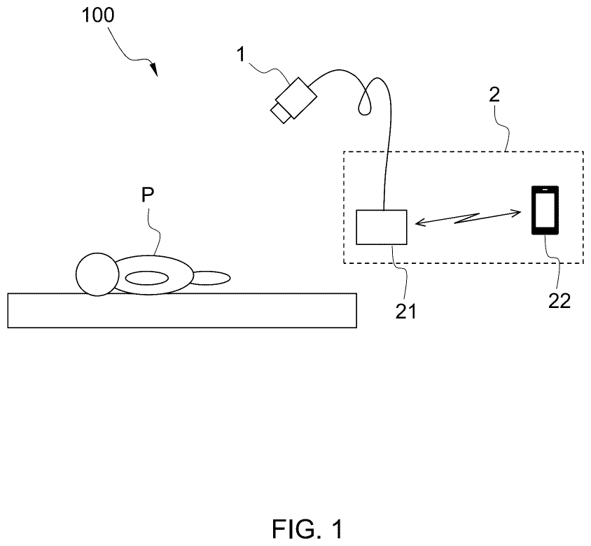

As illustrated in FIG. 1, a sleep depth measurement system 100 according to the present embodiment includes a camera 1 installed in a room such as a bedroom, and an information processing apparatus 2 that receives image data captured by the camera 1 and analyzes the image data to calculate a sleep depth of a subject P.

### Second Configuration of Each Unit

### 1. Camera

The camera 1 includes a lens, a two-dimensional sensor such as a CCD, and the like, and is configured to capture an image of the subject P who is sleeping and output the image data (moving image data). Note that the camera 1 may be of a fixed installation type, may be capable of remotely adjusting an imaging angle and zoom, or may be a depth camera capable of acquiring three-dimensional information.

### 2. Information Processing Apparatus

The information processing apparatus 2 includes a CPU, a GPU, a memory (This includes a volatile memory such as a DRAM and a nonvolatile memory such as an HDD.), an input/output interface, a communication interface, a display, and an input unit (touch panel, mouse, keyboard, and the like). In the present embodiment, as illustrated in the drawing, the information processing apparatus 2 includes a main device 21 connected to the camera 1 so as to be able to communicate with the camera 1 in a wired or wireless manner, and a (mobile) terminal 22 connected to the main device 21 so as to be able to communicate with each other via the Internet or the like.

Note that the information processing apparatus 2 may be, for example, a physically integrated device such as a so-called personal computer, or may further include a server system called a cloud or the like interposed between the main device 21 and the mobile terminal 22. Furthermore, the information processing apparatus 2 may be integrated with the camera 1. In short, the camera 1 and the information processing apparatus 2 are not limited to the physical modes as long as equivalent functions are exhibited.

### 3. Program Configuration

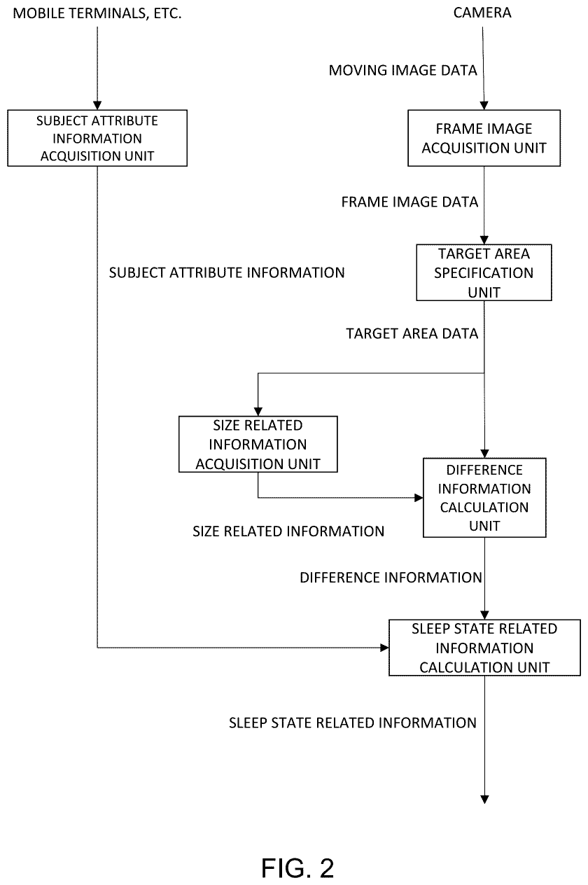

The CPU and its peripheral devices cooperate with each other according to the program stored in the memory, whereby the information processing apparatus 2 functions as a frame image acquisition unit, a target area specification unit, a size related information acquisition unit, a subject attribute information acquisition unit, a difference information calculation unit, a sleep state related information calculation unit, and the like as illustrated in FIG. 2. These components will be described below.

### (1) Frame Image Acquisition Unit

For example, the frame image acquisition unit receives moving image data from the camera 1, extracts or generates a series of still images (frame images) arranged in time series at regular intervals (here, for example, every 0.5 seconds) from the moving image data, and stores the data in a predetermined area of a memory. Note that the camera 1 may generate a frame image, and the frame image acquisition unit may receive and acquire the frame image.

### (2) Target Area Specification Unit

The target area specification unit specifies a target area (ROI) which is a constant image area including the whole or the specific part of the subject P in the frame image acquired by the frame image acquisition unit. The position and range of the target area are common in each frame image.

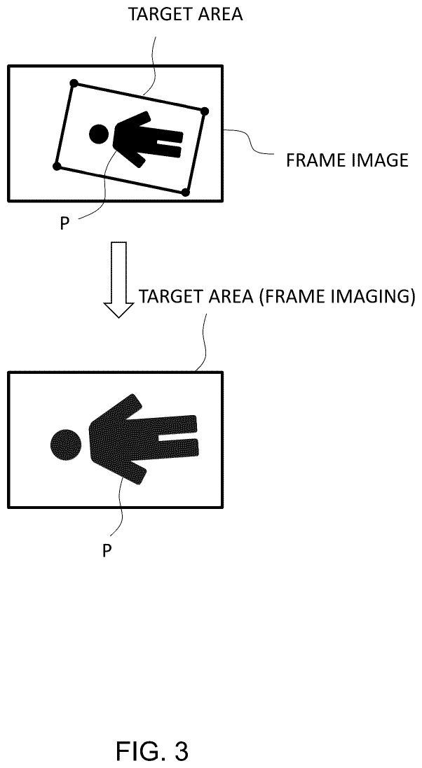

As illustrated in FIG. 3, the target area specification unit specifies, as the target area, a rectangular area set and input by the operator in such a manner of including the entire subject P in the captured image (initial frame image) displayed on the display.

Note that, instead of the setting and inputting by the operator, for example, the imaging area of the subject P may be specified by image recognition based on machine learning such as semantic segmentation, and an appropriate constant shape area such as a rectangle including the imaging area may be automatically set as the target area. The size and position of the target area with respect to the entire frame image may vary for each frame image according to the movement of the subject or the like. However, the size of the target area is preferably set such that the ratio of the size of the target area to the size of the imaged subject P is equal. Furthermore, the target area is not limited to a rectangle or the like, and may have a shape in units of pixels.

### (3) Size Related Information Acquisition Unit

The size related information acquisition unit acquires size related information that is information related to a relative size of all or a specific part of the subject P in the frame image.

Here, the number of area pixels of the target area or the number of length pixels along the predetermined direction of the target area is simply used as the size related information. However, as described above, the image range of the subject P may be specified by an image recognition method or the like based on machine learning such as semantic segmentation, and the number of pixels corresponding to the height of the subject P may be calculated from the image range to calculate more accurate size related information.

### (4) Subject Attribute Information Acquisition Unit

The subject attribute information acquisition unit acquires subject attribute information that is information indicating the attribute of the subject P input by an operator, for example. The subject attribute information here includes age, gender, height, weight, presence or absence of disease, physical condition, and the like.

Note that the subject attribute information can be acquired, for example, in cooperation with an existing personal health record (PHR) without depending on an operator's input. The acquisition timing may be at the time of measurement or analysis as long as the acquisition timing is handled together with the measurement data.

In addition, feature points regarding the body of the subject P may be extracted by an image recognition technique, and the subject attribute information such as age may be estimated from the feature points.

For example, eyeball positions of the subject P are extracted by image recognition, and the pupillary distance is obtained. It has been found that the pupillary distance correlates with the height, physique and age and the degree of development of the child, and the subject attribute information can be estimated.

### (5) Difference Information Acquisition Unit

The difference information acquisition unit calculates, for each frame image, difference information that is information indicating a difference between two frame images at different times, more specifically, a difference between a first frame image (hereinafter, also referred to as a reference frame image) of the two frame images and a frame image (hereinafter, also referred to as a comparison frame image) after a lapse of a certain time interval.

The meaning of the difference information here includes not only the difference information between the entire reference frame image and the entire comparison frame image, but also the difference information between the areas to be partially compared. In this embodiment, difference information for only the target area is calculated in order to reduce calculation load, noise, and the like.

The difference information in this embodiment is, for example, the number of pixels having different pixel values exceeding a predetermined threshold in a case where corresponding pixels are compared in the target area of the reference frame image and the target area of the comparison frame image. The pixel value is represented by a one-dimensional value of only brightness when the frame image is grayscale, and is represented by a three-dimensional value such as RGB or CYK when the frame image is a color image. Note that the difference information may be not only the number of pixels but also a ratio of the number of different pixels to the total number of frames of the frame image. In addition, the movement amount of the feature points (for example, each part of the body and the pupil as described above) may be used as a reference instead of the change in the pixel value.

In addition, in this embodiment, a plurality of (two) time intervals are set as time intervals between frame images for obtaining difference information. Here, the first time interval as one time interval is set to, for example, 0.5 seconds, and the second time interval as the other time interval is set to 3 seconds. Difference information (hereinafter, also referred to as first difference information) between frame images at the first time interval and difference information (hereinafter, also referred to as second difference information) between frame images at the second time interval are sequentially calculated by the difference information calculation unit. Note that in the following description, when it is not necessary to distinguish the first difference information from the second difference information, the first difference information and the second difference information may be simply referred to as difference information.

### (6) Sleep State Related Information Calculation Unit

The sleep state related information calculation unit calculates the sleep state related information, which is information on the sleep state of the subject P, using the difference information and the subject attribute information as an explanatory variable. Note that the sleep state related information mentioned here includes body motion, posture, pulse, respiration, facial expression, sleep depth, occupancy of the sleep depth (time zone ratio of each sleep depth in one sleep), a sleep cycle, and the like.

### Third Operation

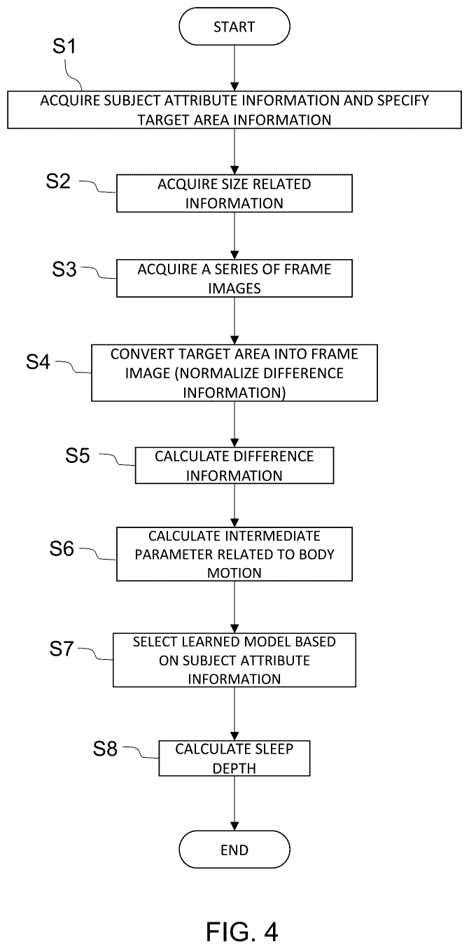

Next, the operation of the sleep depth measurement system 100 will be described in detail with reference to the flowchart of FIG. 4, together with a more detailed description of each unit.

### 1. Initial Setting

First, when the operator activates the sleep depth measurement system 100, the video of the camera 1 is displayed on the display of the terminal 22.

The operator adjusts the field of view, the angle, and the position of the camera 1 so as to capture all of the subject P while checking the image with the terminal 22. This adjustment may be performed by directly operating the camera 1 or remotely from the terminal 22 using the PTZ camera 1.

### 2. Acquire Target Area and Subject Attribute Information

Next, the operator designates and inputs a rectangular target area on the screen of the terminal 22 in such a manner of including the subject P. As a result, target area information indicating the position and range of the target area in the image is specified, and the target area information acquisition unit acquires the target area information and stores the target area information in a predetermined area of the memory (step S1).

The operator inputs the subject attribute information in the terminal 22. The input subject attribute information is acquired by the subject attribute information acquisition unit and stored in a predetermined area of the memory (same step S1).

### 3. Acquire Size Related Information

Next, the size related information acquisition unit acquires the size related information occupied in the frame image of the subject P (step S2). Here, as described above, the number of area pixels of the target area or the number of length pixels along the longitudinal direction of the target area is set as the size related information.

When the measurement is started by the operation of the operator, first, the frame image acquisition unit sequentially extracts or generates data of still images, that is, frame images arranged in time series at regular intervals (here, for example, every 0.5 seconds) from the moving image data transmitted from the camera 1, and stores the frame image data in a predetermined area of the memory (step S3).

### 4. Calculate Difference Information

Next, the difference information calculation unit calculates difference information between each frame image, which serves as a reference, and a frame image after a fixed time interval (step S5). Here, as described above, two time intervals (the first time interval and the second time interval) are set, and two types of difference information (the first difference information and the second difference information) are calculated for each target area of a series of frame images.

At this time, the difference information calculation unit removes obvious noise and errors. As an example, in the first time interval (0.5 seconds), an impossible number of difference pixels (movement) is detected. In such a case, processing such as not counting the frame image as an error is performed.

On the other hand, the sleep state related information calculation unit normalizes the difference information with the size related information. In this embodiment, the normalization of the difference information is performed prior to the calculation of the difference information. (Step S4).

Here, the number of area pixels and the number of vertical and horizontal length pixels of the target area are used as the size related information, and the size ratio between the target area and the subject P on the image is made as equal as possible. Therefore, the target area is set to the size of the image frame. Then, the difference information calculation unit calculates difference information for the target area.

Here, the necessity of normalization of the difference information will be briefly described. In a case where the subject P imaged small in the image is compared with the subject P imaged large in the image, the value of the difference information represented by the number of pixels becomes small in the case of the subject P imaged small in the image even when there is the same body motion. As a result, even when the difference information of 10 pixels under a certain condition corresponds to the movement of one arm, there is a case where the difference information corresponds to the movement of the trunk (rolling over) under another condition, or a case where the difference information is regarded as noise instead of body motion. Then, when the sleep state related information is calculated using such difference information as it is, measurement accuracy is deteriorated. Therefore, such a problem is solved by correcting the difference information by the normalization.

Note that the normalization method is not limited to the above, and calculation processing equivalent thereto may be performed. For example, the processing may be performed after the difference information is calculated. As an example, processing of setting a reference value of the size related information in advance and correcting the difference information by multiplying a reciprocal of a ratio of an acquired value of the size related information to the reference value by a value of the difference information can be performed.

In addition, after the difference information (time-series transition of the body motion amount) to be analyzed is calculated, normalization for setting the entire series of difference information to an average 0 and a variance 1 may be performed, which can then be used as input parameters (explanatory variables) for the learning device. By performing normalization for each piece of data to be analyzed, it is possible to suppress variation of the value of the difference information for each piece of data.

### 5. Calculate Sleep State Information

Next, the sleep state related information calculation unit calculates an intermediate parameter (an example of information obtained by processing the difference information in the claims) based on the corrected (normalized) two types of difference information (step S6). Specifically, it is conceivable that the first difference information (the number of difference pixels) indicates fast body motion, and the second difference information indicates slow body motion. Then, an average value, a variance value, and a time during which the number of difference pixels continues to be equal to or less than a predetermined threshold, and the like in a certain period (a period corresponding to a plurality of epochs, for example, 30 seconds) of the values of the first difference information and the second difference information are calculated as an intermediate parameter. Note that the intermediate parameter is also included in the sleep state related information.

On the other hand, the sleep state related information calculation unit selects a learned model based on the subject attribute information (step S7).

The learned model is an algorithm generated in advance by machine learning to output the sleep depth when the difference information and/or the intermediate parameter is input. Here, the learning data (the difference information and/or the intermediate parameter) is divided into a plurality of pieces according to the value of the subject attribute information, and machine learning is executed for each piece of the learning data to generate a learned model for each value of the subject attribute information.

More specifically, in this embodiment, age is used as the subject attribute information, and a learned model is generated in advance for each age group. The age group includes 0 year old to 2 years old, 2 years old to 8 years old, and the like.

Next, the sleep state related information calculation unit calculates the sleep depth by inputting the intermediate parameter to the learned model (step S8).

### Fourth Other Embodiments

Note that the present invention is not limited to the above embodiments.

For example, the body motion may be calculated using the variation of the key point coordinates of the body estimated by the skeleton estimation.

The sleep state related information may be measured only by a specific part such as a face, a hand, or a foot instead of the entire body of the subject P.

Although the body motion information is calculated from the difference information between the camera moving image frames, the body motion information may be calculated from the variation of the output value of a contact sensor or the like at every predetermined time.

In the above embodiment, the size related information is set by the number of pixels of the area imaged in the image of the subject P. However, the distance to the subject P may be corrected as a parameter. For example, in a case where an image is captured obliquely, the distance from the camera 1 is shorter on the front side of the image, and the distance from the camera 1 becomes longer as going deeper. Therefore, even for the same subject P, when the subject P is imaged on the front side of the image, the subject P is imaged larger, and when the subject P is imaged on the back side, the subject P is imaged smaller. Therefore, the value of the size related information may be corrected depending on the position of the subject P in the image. Specifically, in a case where the subject P is on the front side, correction is performed so as to reduce the number of imaging pixels, and in a case where the subject P is on the back side, the opposite is performed.

In this case, the size related information may be calculated for each image frame. For example, when the subject P is a child, the movement range due to sleeping posture is large, and a change in the relative positional relationship (particularly, distance) with the camera 1 occurring during sleeping cannot be ignored.

In addition, a body part specification unit that is a program for specifying a body part of the subject P may be provided.

For example, when the operator designates the face area with a tap or the like, the body part specification unit can detect the face with “Premise that there is a face in the designated area”.

The acquisition of the attribute information may also be estimated from the size related information. For example, it is possible to estimate attribute information such as height and age from the pupillary distance (or the interocular distance) and the head size of the subject P. Through this step, the learned model may be selected according to the size related information.

One of the learned models may be selected according to age or the like, or a plurality of learned models may be selected to perform ensemble learning. For example, in a case where the growth of the subject P is faster than the average, it is conceivable to use both a learned model based on age and a learned model based on body shape (height, weight).

As the user interface, in addition to the input items of age and body shape, there may be an input item such as “body shape of subject P is larger/smaller than average”. The body shape of the subject P may be automatically determined by, for example, linking the body shape of the subject P with the data of the body growth curve.

Regarding the “select a plurality of learned models”, learning devices (models) having different pieces of learning data may be prepared by the same machine learning method, or learning devices (models) created by different machine learning methods may be used.

A machine learning method in which estimation accuracy of the sleep state related information becomes high may be different depending on attribute information such as age. For example, in and after infant stage in which the sleep rhythm is acquired, the characteristic of body motion appearance according to the sleep stage becomes clear. Since there is a clear feature for each sleep stage, it is compatible with sleep depth determination by Extra Tree (majority method). On the other hand, for the estimation of the sleep depth in infancy when the sleep rhythm is not acquired, the correct answer rate is higher when the sleep depth is estimated by the learning model using the neural network.

In addition, the learned model learned in advance may be updated with the information of the subject P. For example, it is conceivable that the parent of the subject P annotates the sleep depth information. All the learning data may be prepared for the measurement subject. In this case, it is preferable to increase the learning data by data augmentation.

In the above embodiment, the sleep depth is measured by calculating the difference information after the series of frame images is acquired, but the sleep depth may be calculated simultaneously in parallel while capturing the frame images as in streaming.

In the above configuration, the sleep state related information is calculated based on the image data of the subject, but the present invention is not limited to the image data.

For example, biological information may be extracted from the subject, time variation information of the biological information may be calculated, and the sleep state related information may be calculated according to the subject attribute information using the time variation information or information obtained by processing the time variation information as an explanatory variable.

The biological information mentioned here is, for example, one or more of body motion, a respiration rate (or its dispersion), an amplitude of a respiration waveform, a heart rate, a pulse rate, a pulse interval, and a time variation of the pulse interval. Examples of a biological information sensor for measuring the biological information include an electromagnetic wave sensor that detects a heartbeat, a pulse wave, or the like from a transmission amount or a reflection amount of an electromagnetic wave such as infrared rays, and a pressure sensor that detects a load change or a load movement. As a form of the biological information sensor, a sheet type, a wearable type, a microwave radar type, or the like may be used.

The biological information may be extracted at a specific part by detecting a landmark in the frame image, determining the specific part with reference to the landmark.

When the biological information is, for example, respiratory information (respiratory rate or amplitude of respiratory waveform), the landmark can be a face or a neck. The position of the landmark may be based on an operator's input, or may be performed by machine learning using a learned model that outputs the position of the landmark with a frame image as an input.

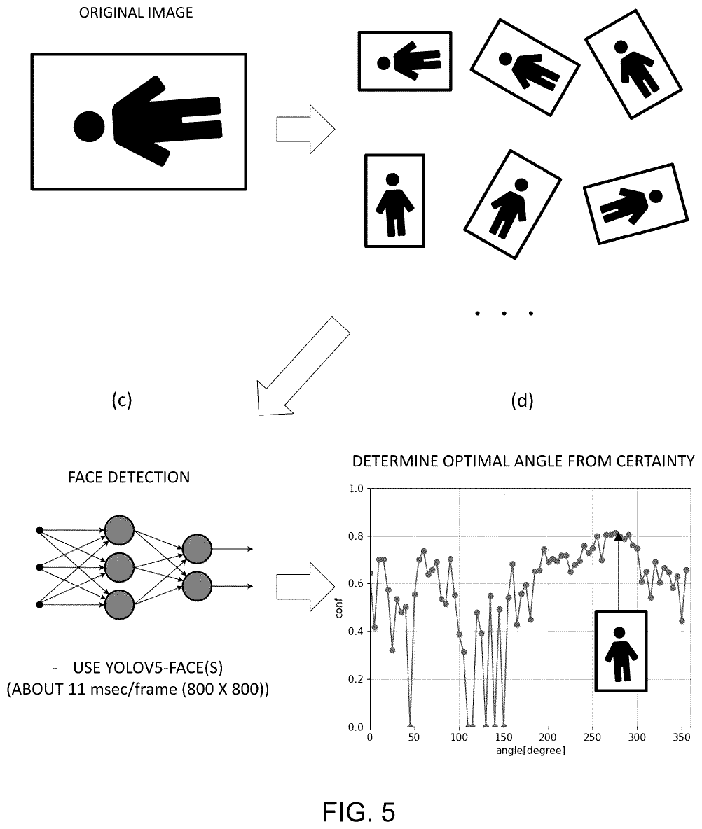

Specific examples thereof are illustrated in FIGS. 5(a) to 5(d). Here, while the frame image (as illustrated in FIG. 5(a)) is rotated in the plane at a constant angle (as illustrated in FIG. 5 (b)), the landmarks are estimated at a plurality of angles by machine learning (as illustrated in FIG. 5 (c)), and the detection result of the landmarks at the angle with the highest certainty is adopted (as illustrated in FIG. 5 (d)).

When the landmark is the face and the biological information is the respiratory information, the thoracoabdominal area is determined from the position of the face area. Respiratory information may be extracted as biological information from the estimated motion of the thoracoabdominal area. However, when the face is inclined with respect to the body axis, the actual position of the thoracoabdominal area may deviate from the determined position. Therefore, the periphery of the determined position of the thoracoabdominal area may be determined as the target area, and the respiratory information as the biological information may be extracted in the target area.

By extracting the biological information in the thoracoabdominal area or the target area, it is possible to exclude the movement of another part that becomes noise.

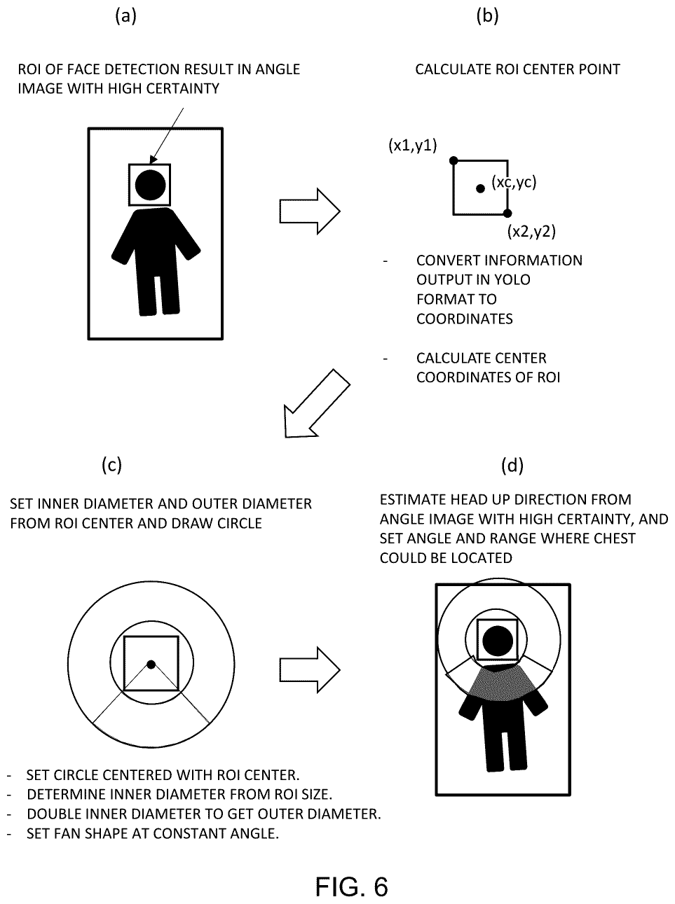

The shape of the target area may be set in a donut shape in a certain angular range with the determined position of the landmark as the rotation center. A specific example thereof will be described with reference to FIG. 6. The ROI (area of interest) of the face detection result in the angle image with high certainty is calculated (FIG. 6(a)), and the center point is calculated (FIG. 6(b)). Then, a double concentric circle is drawn from the center point, and a fan shape at a constant angle is set from the donut area surrounded by the double concentric circle (FIG. 6 (c)). This shape is applied to an angle image with high certainty, and the fan-shaped range is set to a range in which the chest (abdomen) can exist (FIG. 6 (d)).

By limiting the target area in this manner, there is an advantage that the amount of calculation can be reduced and the movement of another part that becomes noise can be excluded.

Note that, when the above-described camera is considered together with the information processing apparatus that processes the image data, it can also be said that the camera has a function as a biological information sensor that detects biological information.

In addition, since the time variation information or the information obtained by processing the time variation information is the time variation of the biological information, it can be said that the difference information or the information obtained by processing the difference information is also included in the time variation information or the like.

In addition, the present invention is not limited to the embodiment and the illustrated example, and can be modified without departing from the gist of the present invention, for example, by combining some of the embodiments and the modified examples.

### Fifth Summary

Features of the above-described embodiments are summarized as follows.

[1]

A sleep state measurement system including: a frame image acquisition unit that acquires frame images including a subject during sleep in time series; a difference information calculation unit that calculates difference information that is information indicating a difference between two frame images at different times; a subject attribute information acquisition unit that acquires subject attribute information that is information indicating an attribute of the subject; and a sleep state related information calculation unit that calculates sleep state related information that is information related to a sleep state of the subject using the difference information and the subject attribute information as an explanatory variable.

In such a case, since the sleep state is measured using the subject attribute information such as age as an explanatory variable in addition to being able to perform measurement in a natural sleep state without a burden on the subject, the measurement accuracy can be improved as compared with the related art.

[2]

The sleep state measurement system according to [1], wherein the sleep state related information calculation unit selects one or a plurality of learned models according to content of the subject attribute information from among a plurality of learned models, and calculates the sleep state related information by giving the difference information to the learned models.

In such a case, since the present sleep state measurement system can be realized by generating a plurality of types of learned models by machine learning, it is possible to ensure high feasibility and measurement accuracy. In particular, the improvement of the measurement accuracy can be promoted by increasing the learning data by the data augmentation.

[3]

The sleep state measurement system according to [1] or [2], wherein the age of the subject is used as the subject attribute information.

In such a case, since the change pattern of the sleep depth (sleep cycle, occupancy, etc.) can vary depending on children, infants, general adults, and elderly people, it is possible to expect a remarkable improvement in the measurement accuracy by changing the algorithm of the sleep state calculation such as generating the learned model according to the value using the age (or the age group) as the subject attribute information.

[4]

The sleep state measurement system according to any one of [1] to [3], further including a size related information acquisition unit that acquires size related information that is information related to a relative size of all or a specific part of a subject in the frame image, wherein the sleep state related information calculation unit calculates the sleep state related information also using the size related information as an explanatory variable.

[5]

The sleep state measurement system according to [4], wherein the sleep state related information calculation unit normalizes the difference information by the size related information, and calculates the sleep state related information based on the normalized difference information and the subject attribute information.

In such a case, it is possible to stabilize the difference information and the reference of the body motion amount calculated based on the difference information regardless of the size of the subject imaged in the image, and thus, it is possible to improve the measurement accuracy of the sleep state related information such as the sleep depth.

[6]

The sleep state measurement system according to [4] or [5], further including a target area specification unit that specifies a target area having a predetermined shape including all or a specific part of a subject in the frame image, wherein the difference information calculation unit is configured to calculate difference information between the target areas in two frame images, and the size related information acquisition unit acquires, as the size related information, the number of area pixels of the target area or the number of length pixels of the target area in a predetermined direction.

In such a case, the configuration of [4] or [5] can be easily realized.

[7]

The sleep state related information includes a sleep depth, a sleep cycle, and an occupancy rate.

In such a case, the effect of this configuration becomes more remarkable.

[8]

The sleep state measurement system according to [1], further including: an extraction unit that extracts biological information from a subject during sleep;
a calculation unit that calculates time variation information of the biological information;
a subject attribute information acquisition unit that acquires subject attribute information that is information indicating an attribute of the subject; and
a sleep state related information calculation unit that calculates sleep state related information that is information related to a sleep state of the subject, using the time variation information or information obtained by processing the time variation information as an explanatory variable according to the subject attribute information.

In such a case, the sleep state related information can be calculated from the biological information.

[9]

The sleep state measurement system according to [8], wherein the biological information includes one or more of body motion, a respiration rate, a variance of the respiration rate, an amplitude of a respiration waveform, a heart rate, a pulse rate, a pulse interval, and a time variation of the pulse interval.

By using such biological information, the measurement accuracy of the sleep state can be improved.

[10]

The sleep state measurement system according to any one of [1] to [9], wherein the extraction unit detects a landmark in the frame image, and determines a specific part with reference to the landmark, and extracts the biological information in the specific part.

By limiting the target area in this manner, the amount of calculation can be reduced and the movement of another part that becomes noise can be excluded.

[11]

The sleep state measurement system according to any one of [1] to [9], wherein the extraction unit detects a landmark in the frame image, determines a specific part with reference to the landmark, determines a periphery of the specific part as a target area, and extracts the biological information in the target area.

In this way, the target area can also be limited, and, as mentioned above, the amount of calculation can be reduced and the movement of another part that becomes noise can be excluded.

[12]

The sleep state measurement system according to [11], wherein
the landmark is a face of a subject,
the specific part is a chest and abdomen, and
the biometric information is respiratory information.

In such a case, the measurement accuracy of the sleep state can be improved.

[13]

A sleep state measurement system including: an extraction unit that extracts biological information from a subject during sleep;
a calculation unit that calculates time variation information of the biological information;
a subject attribute information acquisition unit that acquires subject attribute information that is information indicating an attribute of the subject; and
a sleep state related information calculation unit that calculates sleep state related information that is information related to a sleep state of the subject, using the time variation information or information obtained by processing the time variation information as an explanatory variable according to the subject attribute information.

In such a case, the same functions and effects as those of the configuration of [1] described above can be obtained.

### INDUSTRIAL APPLICABILITY

Since the sleep state is measured using the subject attribute information such as age as an explanatory variable in addition to being able to perform measurement in a natural sleep state without a burden on the subject, the measurement accuracy can be improved as compared with the related art.

### REFERENCE SIGNS LIST

100 sleep state measurement system
1 camera
2 information processing apparatus
P subject

1. A sleep state measurement system comprising: a frame image acquisition unit that acquires frame images including a subject during sleep in time series;
a difference information calculation unit that calculates difference information that is information indicating a difference between two frame images at different times;
a subject attribute information acquisition unit that acquires subject attribute information that is information indicating an attribute of the subject; and
a sleep state related information calculation unit that calculates sleep state related information that is information related to a sleep state of the subject, using the difference information or information obtained by processing the difference information as an explanatory variable according to the subject attribute information.

2. The sleep state measurement system according to claim 1, wherein the sleep state related information calculation unit selects one or a plurality of learned models according to content of the subject attribute information from among a plurality of learned models that are identical to each other or different from each other, and calculates the sleep state related information by giving the difference information to the learned models.

3. The sleep state measurement system according to claim 2, wherein an age of a subject is used as the subject attribute information.

4. The sleep state measurement system according to any one of claim 1 to claim 3, further comprising: a size related information acquisition unit that acquires size related information that is information related to a relative size of all or a specific part of a subject in the frame image, wherein
the sleep state related information calculation unit calculates the sleep state related information also using the size related information as an explanatory variable.

5. The sleep state measurement system according to claim 4, wherein the sleep state related information calculation unit normalizes the difference information by the size related information, and calculates the sleep state related information based on the difference information normalized and the subject attribute information.

6. The sleep state measurement system according to claim 4, further comprising: a target area specification unit that specifies a target area having a predetermined shape including all or a specific part of a subject in the frame image, wherein
the difference information calculation unit is configured to calculate difference information between the target area and the target area in two frame images, and
the size related information acquisition unit acquires, as the size related information, a number of area pixels of the target area or a number of length pixels of the target area in a predetermined direction.

7. The sleep state measurement system according to claim 1, wherein the sleep state related information includes a sleep depth.

8. The sleep state measurement system according to claim 1, further comprising: an extraction unit that extracts biological information from a subject during sleep; and
a calculation unit that calculates time variation information of the biological information, wherein
the sleep state related information calculation unit calculates sleep state related information that is information related to a sleep state of the subject, using the difference information or information obtained by processing the difference information and the time variation information or information obtained by processing the time variation information as an explanatory variable according to the subject attribute information.

9. The sleep state measurement system according to claim 8, wherein the biological information includes one or more of body motion, a respiration rate, a variance of the respiration rate, an amplitude of a respiration waveform, a heart rate, a pulse rate, a pulse interval, and a time variation of the pulse interval.

10. The sleep state measurement system according to claim 8, wherein the extraction unit detects a landmark in the frame image, and determines a specific part with reference to the landmark, and extracts the biological information in the specific part.

11. The sleep state measurement system according to claim 8, wherein the extraction unit detects a landmark in the frame image, determines a specific part with reference to the landmark, determines a periphery of the specific part as a target area, and extracts the biological information in the target area.

12. The sleep state measurement system according to claim 11, wherein
the landmark is a face of a subject,
the specific part is a chest and abdomen, and
the biological information is respiratory information.

13. A sleep state measurement method comprising: acquiring frame images including a subject during sleep in time series;
calculating difference information that is information indicating a difference between frame images at different times based on a comparison result of the frame images at the different times;
acquiring subject attribute information that is information indicating an attribute of the subject; and
calculating sleep state related information that is information related to a sleep state of the subject, using the difference information or information obtained by processing the difference information as an explanatory variable according to the subject attribute information.

14. A sleep state measurement program that causes a computer to function as: a frame image acquisition unit that acquires frame images including a subject during sleep in time series;
a difference information calculation unit that calculates difference information that is information indicating a difference between frame images at different times based on a comparison result of the frame images at the different times,
a subject attribute information acquisition unit that acquires subject attribute information that is information indicating an attribute of the subject; and
a sleep state related information calculation unit that calculates sleep state related information that is information related to a sleep state of the subject, using the difference information or information obtained by processing the difference information as an explanatory variable according to the subject attribute information.

15. A sleep state measurement system comprising: an extraction unit that extracts biological information from a subject during sleep;
a calculation unit that calculates time variation information of the biological information;
a subject attribute information acquisition unit that acquires subject attribute information that is information indicating an attribute of the subject; and
a sleep state related information calculation unit that calculates sleep state related information that is information related to a sleep state of the subject, using the time variation information or information obtained by processing the time variation information as an explanatory variable according to the subject attribute information.
